(19) United States
(12) Patent Application Publication
(10) Pub. No.: US 2026/0026871 A1
(43) Pub. Date: Jan. 29, 2026

(54) ULTRASONIC TREATMENT TOOL

(71) Applicant: OLYMPUS MEDICAL SYSTEMS CORP., Tokyo (JP)

(72) Inventors: Tsubasa NIIYAMA, Hachioji-shi (JP); Marina ASAHINA, Sagamihara-shi (JP); Keisuke NAGAO, Hachioji-shi (JP); Kaito SATO, Tachikawa-shi (JP)

(21) Appl. No.: 19/008,200

(22) Filed: Jan. 2, 2025

Related U.S. Application Data
(63) Continuation of application No. PCT/JP2024/026714, filed on Jul. 25, 2024.

(51) Int. Cl. A61B 18/14; A61B 18/00; A61B 90/00

(52) U.S. Cl. CPC A61B 18/1445; A61B 90/03; A61B 2018/00077; A61B 2018/00083; A61B 2018/00107; A61B 2018/00178; A61B 2018/00184; A61B 2018/00601; A61B 2018/00994; A61B 2018/1452; A61B 2090/034

(57) ABSTRACT

An ultrasonic treatment tool includes: an ultrasonic blade configured to supply ultrasonic vibration and high-frequency current to a body tissue; a jaw configured to open and close with respect to the ultrasonic blade; a holder that is supported by the jaw, is made of a first material having electrical insulation properties, and includes an electrode supplying the high-frequency current and a protruding portion protruding toward the ultrasonic blade, the electrode being disposed on a face that is included in the holder and that faces the ultrasonic blade; and an abutting portion that is disposed in the holder and is made of a first resin material, the abutting portion being configured to abut against the ultrasonic blade when the jaw closes with respect to the ultrasonic blade.

### CROSS REFERENCES TO RELATED APPLICATIONS

This application is a continuation of International Application No. PCT/JP2024/026714, filed on Jul. 25, 2024, the entire contents of which are incorporated herein by reference.

### BACKGROUND

### 1. Technical Field

The present disclosure relates to an ultrasonic treatment tool.

### 2. Related Art

In the related art, an ultrasonic treatment tool is known that supplies ultrasonic vibrations and a high-frequency current as the treatment energy to the part to be treated in the body tissue (hereinafter, referred to as the treatment target), and accordingly treats the treatment target (for example, refer to Japanese Unexamined Patent Application Publication (Translation of PCT application) No. 2019-509775).

The ultrasonic treatment tool disclosed in Japanese Unexamined Patent Application Publication (Translation of PCT application) No. 2019-509775 includes an ultrasonic blade, a jaw, and an abutting portion explained below. The ultrasonic blade supplies ultrasonic vibrations and a high-frequency current to the treatment target. The jaw opens and closes with respect to the ultrasonic blade. Moreover, the jaw has an electrode disposed therein for supplying a high-frequency current to the treatment target. The abutting portion is made of a resin material, and is disposed in the jaw. When the jaw closes with respect to the ultrasonic blade, the abutting portion abuts against the ultrasonic blade.

### SUMMARY

In some embodiments, an ultrasonic treatment tool includes: an ultrasonic blade configured to supply ultrasonic vibration and high-frequency current to a body tissue; a jaw configured to open and close with respect to the ultrasonic blade; a holder that is supported by the jaw, is made of a first material having electrical insulation properties, and includes an electrode supplying the high-frequency current and a protruding portion protruding toward the ultrasonic blade, the electrode being disposed on a face that is included in the holder and that faces the ultrasonic blade; and an abutting portion that is disposed in the holder and is made of a first resin material, the abutting portion being configured to abut against the ultrasonic blade when the jaw closes with respect to the ultrasonic blade.

In some embodiments, an ultrasonic treatment tool includes: an ultrasonic blade supplying ultrasonic vibration and high-frequency current to a body tissue; a jaw that is made of a first material having electrical insulation properties and includes an electrode supplying the high-frequency current and a protruding portion protruding toward the ultrasonic blade, the jaw being configured to open and close with respect to the ultrasonic blade, the electrode being disposed on a face that is included in the jaw and that faces the ultrasonic blade; and an abutting portion that is disposed in the jaw, and is made of a first resin material, the abutting portion being configured to abut against the ultrasonic blade when the jaw closes with respect to the ultrasonic blade.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an ultrasonic treatment tool according to a first embodiment;

FIGS. 2 and 3 are diagrams for explaining a configuration of the front end portion in the ultrasonic treatment tool;

FIG. 4 is a diagram for explaining a configuration of a jaw;

FIG. 5 is a diagram for explaining a configuration of a holder;

FIG. 6 is a partially expanded view of the proximal end side of the holder;

FIG. 7 is a diagram illustrating the positional relationship among the constituent elements in the case in which the front end portion of the treatment tool is twisted; and

FIG. 8 is a diagram illustrating an ultrasonic treatment tool according to a second embodiment.

### DETAILED DESCRIPTION

Illustrative embodiments are described below with reference to the accompanying drawings. However, the disclosure is not limited by the embodiments described below. Moreover, in the drawings, identical constituent elements are referred to by the same reference numerals.

### First Embodiment

### Overall Configuration of Treatment System

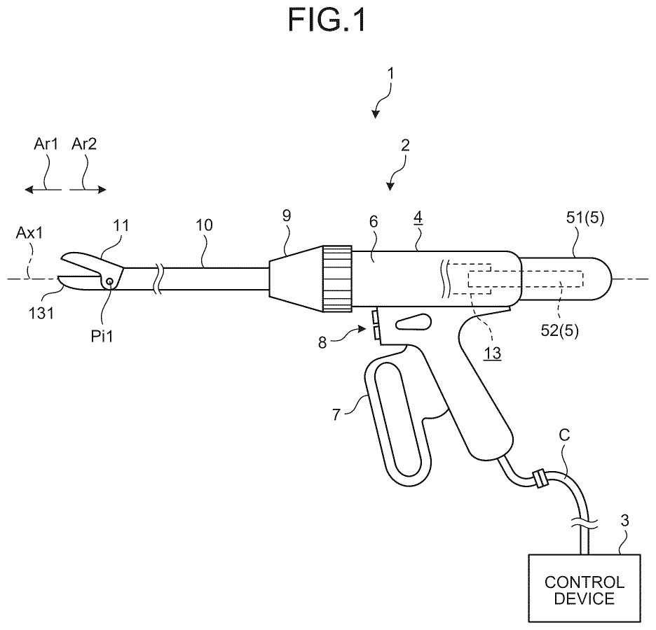

FIG. 1 is a diagram illustrating an ultrasonic treatment tool according to a first embodiment. A treatment system 1 applies treatment energy to the part to be treated in the body tissue (hereinafter, referred to as the treatment target) and accordingly treats the treatment target. The treatment energy according to the first embodiment represents ultrasonic energy and high-frequency energy. Meanwhile, the treatment that can be carried out by the treatment system 1 according to the first embodiment includes solidification (sealing) of the treatment target or dissection of the treatment target. Alternatively, solidification and dissection can be performed in a simultaneous manner. As illustrated in FIG. 1, the treatment system 1 includes a treatment tool 2 and a control device 3.

### Configuration of Treatment Tool

In the following explanation, along a central axis Ax1 of an outer pipe 10 (see FIG. 1), one side of an outer pipe 10 is referred to as a front end side Ar1 and the other side of an outer pipe 10 is referred to as a proximal end side Ar2. Moreover, the term “width direction” mentioned below represents the direction orthogonal to the opening-closing direction of a jaw 11 with respect to the central axis Ax1 and a treatment portion 131, and implies the direction orthogonal to the plane in which FIG. 1 is illustrated.

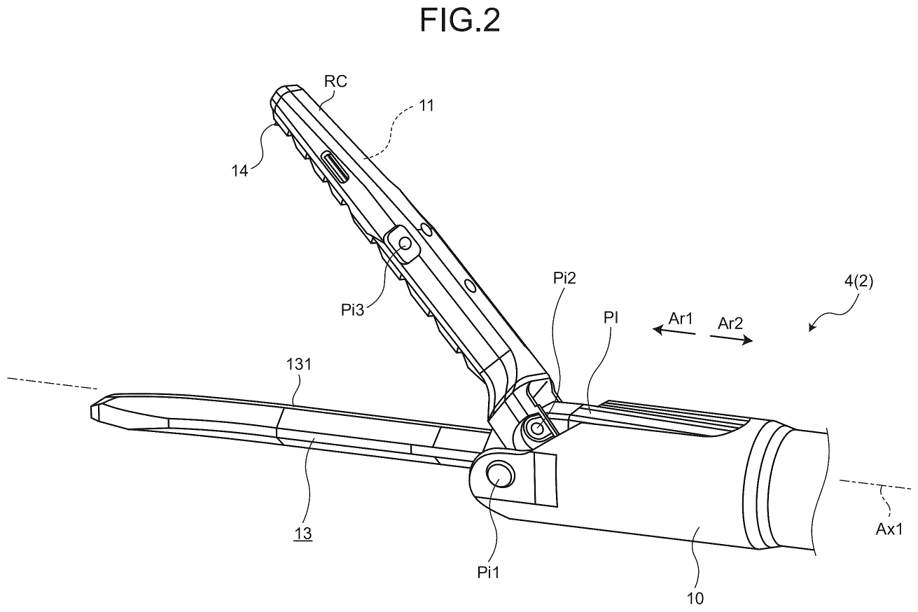

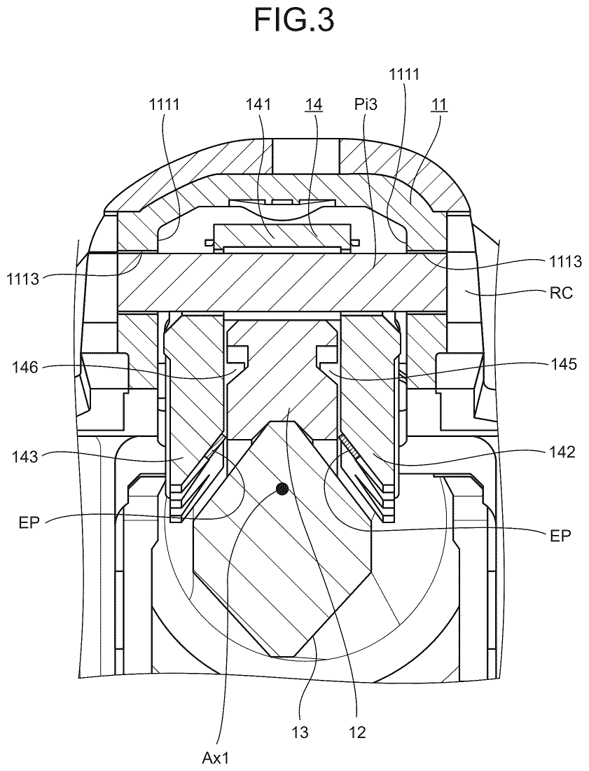

FIGS. 2 and 3 are diagrams for explaining a configuration of the front end portion in the treatment tool 2. More particularly, FIG. 2 is a perspective view of the front end portion in the treatment tool 2. FIG. 3 is a cross-sectional view obtained as a result of cutting the front end portion of the treatment tool 2 by the plane orthogonal to the central axis Ax1.

The treatment tool 2 represents an ultrasonic treatment tool. The treatment tool 2 applies ultrasonic energy and high-frequency energy to the treatment target, and accordingly treats the treatment target. As illustrated in FIG. 1, the treatment tool 2 includes a handpiece 4 and an ultrasonic transducer 5.

As illustrated in FIGS. 1 to 3, the handpiece 4 includes a fixed handle (see FIG. 1), an operation handle 7 (see FIG. 1), a switch 8 (see FIG. 1), a rotation knob 9 (see FIG. 1), an outer pipe 10 (see FIGS. 1 and 2), the jaw 11, an abutting portion 12 (see FIG. 3), an ultrasonic blade 13, and a holder 14 (see FIGS. 2 and 3).

A fixed handle 6 supports the entire treatment tool 2 and represents the portion that is gripped by the operator (user) such as a technician.

The operation handle 7 is attached to the fixed handle 6 in a movable manner, and receives opening-closing operations for the jaw 11 as performed by the operator such as a technician.

The switch 8 is disposed in an exposed state on the outside of the fixed handle 6, and receives treatment operations performed by the operator such as a technician.

The rotation knob 9 has a substantially cylindrical shape that is coaxial to the central axis Ax1, and is disposed on the front end side Ar1 of the fixed handle 6. The rotation knob 9 receives rotation operations performed by the operator such as a technician. In response to a rotation operation, the rotation knob 9 rotates with respect to the fixed handle 6 around the rotation axis Ax1. Moreover, due to the rotation of the rotation knob 9; the outer pipe 10, the jaw 11, the holder 14, the abutting portion 12, and the ultrasonic blade 13 rotate around the central axis Ax1.

The outer pipe 10 is a tubular pipe and represents a pipe. In the first embodiment, the outer pipe 10 is a cylindrical pipe made of an electrically conductive material such as a metal.

In the outer pipe 10, at the end portion on the front end side Ar1, a first pin Pi1 (see FIGS. 1 and 2) that has a columnar shape extending in the direction orthogonal to the plane in which FIG. 1 is illustrated, that engages with the jaw 11, and that supports the jaw 11 in a pivotable manner is fixed. In the first embodiment, the first pin Pi1 is made of an electrically conductive material such as a metal.

The outer periphery of the outer pipe 10 is covered by an outer tube (not illustrated) that is made of an electrically insulating material. Inside the outer pipe 10, a tubular inner pipe PI (see FIG. 2) is inserted that reciprocates along the longitudinal direction of the outer pipe 10 according to an opening-closing action of the operation handle 7 performed by the operator such as a technician. Moreover, at the end portion of the inner pipe P1 on the front end side Ar1, a second pin Pi2 (see FIG. 2) that has a columnar shape extending in the direction orthogonal to the plane in which FIG. 1 is illustrated and that engages with the jaw 11 is fixed. In the first embodiment, in FIG. 2, with reference to the first pin Pi1, the second pin Pi2 is disposed on the upper side (i.e., the side on which a jaw main body 111 is disposed with respect to the treatment portion 131).

The jaw 11 is connected to the outer pipe 10 using the first pin Pi1. Moreover, the jaw 11 is connected to the inner pipe PI using the second pin Pi2. Thus, in tandem with the reciprocation of the inner pipe PI according to an opening-closing action of the operation handle 7 performed by the operator such as a technician, the jaw 11 pivots with respect to the outer pipe 10 around the first pin Pi1. It results in the opening and closing of the jaw 11 with respect to the treatment portion 131 representing the end portion of the ultrasonic blade 13 on the front end side, and the treatment target can be gripped in between the jaw 11 and the treatment portion 131.

Meanwhile, the treatment tool 2 can be configured as a close-by-pressing type tool or as a close-by-pulling type tool.

The treatment tool 2 of the close-by-pressing type has the following configuration.

In tandem with the movement of the inner pipe PI toward the front end side Ar1, the jaw 11 pivots around the first pin Pi1 and in the direction approaching the treatment portion 131. That is, the jaw 11 closes with respect to the treatment portion 131. Moreover, in tandem with the movement of the inner pipe PI toward the proximal end side Ar2, the jaw 11 pivots around the first pin Pi1 and in the direction moving away from the treatment portion 131. That is, the jaw 11 opens with respect to the treatment portion 131.

The treatment tool 2 of the close-by-pulling type has the following configuration.

In tandem with the movement of the inner pipe PI toward the proximal end side Ar2, the jaw 11 pivots around the first pin Pi1 and in the direction approaching the treatment portion 131. That is, the jaw 11 closes with respect to the treatment portion 131. Moreover, in tandem with the movement of the inner pipe PI toward the front end side Ar1, the jaw 11 pivots around the first pin Pi1 and in the direction moving away from the treatment portion 131. That is, the jaw 11 opens with respect to the treatment portion 131.

Meanwhile, the configuration can be such that the outer pipe 10 reciprocates according to an opening-closing action of the operation handle 7. In that case, in tandem with the reciprocation of the outer pipe 10, the jaw 11 pivots around the second pin Pi2, and opens and closes with respect to the treatment portion 131.

Regarding the detailed configuration of the jaw 11, the explanation is given later in a section “Regarding configuration of jaw”.

The holder 14 extends in the direction orthogonal to the plane in which FIG. 3 is illustrated and is supported by a third pin Pi3 (see FIG. 2), which is fixed to the jaw 11, in a swingable manner around the central axis of the third pin Pi3 and in a rotatable manner with respect to the jaw 11. As a result of making the holder 14 swingable around the central axis of the third pin Pi3, when the treatment target is gripped in between the jaw 11 and the treatment portion 131, the position for applying the strongest force onto the treatment target is not set on the proximal end side Ar2 of the jaw 11 but is set substantially at the center of the jaw 11 in the longitudinal direction. Thus, a substantially even force is applied onto the treatment target that is gripped in between the jaw 11 and the treatment portion 131. In the first embodiment, the third pin Pi3 is made of an electrically conductive material such as a metal.

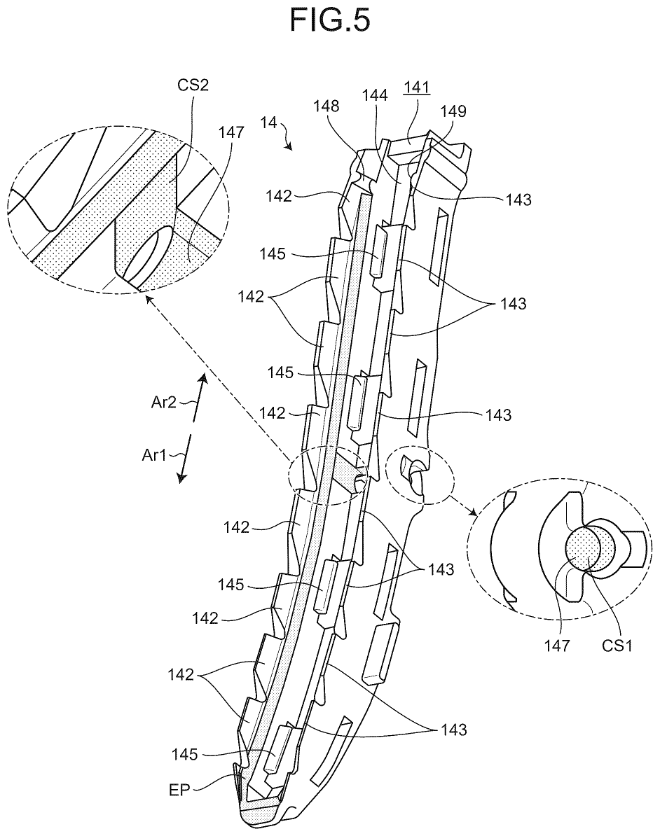

The holder 14 includes an electrode EP, a first conducting surface CS1, and a second conducting surface CS2 (see FIG. 5).

Regarding the detailed configuration of the holder 14, the explanation is given later in a section “Regarding configuration of holder”. Moreover, regarding the configuration of the electrode EP, the first conducting surface CS1, and the second conducting surface CS2; the explanation is given later in a section “Regarding configuration of electrode, first conducting surface, and second conducting surface”.

The abutting portion 12 is made of a first resin material having electrical insulation properties and biocompatibility. The first resin material is, for example, polytetrafluoroethylene (PTFE). The abutting portion 12 has a substantially cuboid shape extending along the longitudinal direction of the jaw 11 and the holder 14. As illustrated in FIGS. 2 and 3, the abutting portion 12 is fixed to a face that is included in a holder main body 141 and that is located on the side of the treatment portion 131; and, when the jaw 11 closes with respect to the treatment portion 131, abuts against the treatment portion 131. The abutting portion 12 has the function by which, when dissection of the treatment target due to ultrasonic vibrations is completed, the treatment portion 131 undergoing ultrasonic vibrations is prevented from colliding with the jaw 11 and getting damaged.

The ultrasonic blade 13 is made of an electrically conductive material and has an elongated shape extending along the central axis Ax1. In the state in which the treatment portion 131 is protruding toward the outside, the ultrasonic blade 13 is inserted inside the inner pipe PI. At that time, as illustrated in FIG. 1, the end portion of the ultrasonic blade 13 on the proximal end side Ar2 gets mechanically connected to an ultrasonic vibrator 52 that constitutes the ultrasonic transducer 5. The ultrasonic blade 13 transmits the ultrasonic vibrations, which are generated by the ultrasonic transducer 5, from the end portion of the ultrasonic blade 13 on the proximal end side Ar2 to the treatment portion 131. The ultrasonic vibrations are longitudinal vibrations occurring along the central axis Ax1. Meanwhile, the outer periphery of the ultrasonic blade 13 excluding the treatment portion 131 is covered by an inner tube that is made of an electrically insulating material.

As illustrated in FIG. 1, the ultrasonic transducer 5 includes a TD (transducer) case 51 and the ultrasonic vibrator 52.

The TD case 51 supports the ultrasonic vibrator 52 and is connected to the fixed handle 6 in a detachably attachable manner.

Under the control performed by the control device 3, the ultrasonic vibrator 52 generates ultrasonic vibrations. In the first embodiment, the ultrasonic vibrator 52 is configured using a bolt-clamped Langevin type transducer (BLT).

### Configuration of Control Device

The control device 3 comprehensively controls the operations of the treatment tool 2 via an electric cable C (see FIG. 1). More particularly, via the electric cable C, the control device 3 detects a treatment operation that is performed by the operator, such as a technician, using the switch 8. When a treatment operation is detected, the control device 3 applies the treatment energy to the treatment target, which is being gripped in between the jaw 11 and the treatment portion 131, via the electric cable C. That is, the control device 3 treats the treatment target.

For example, at the time of applying the ultrasonic energy to the treatment target, the control device 3 supplies a driving power to the ultrasonic vibrator 52 via the electric cable C. As a result, the ultrasonic vibrator 52 generates longitudinal vibrations occurring along the central axis Ax1 (i.e., generates ultrasonic vibrations). According to those longitudinal vibrations, the treatment portion 131 vibrates at the desired amplitude. Then, the ultrasonic vibrations are supplied from the treatment portion 131 to the treatment target, which is being gripped in between the jaw 11 and the treatment portion 131. Thus, the ultrasonic energy is applied from the treatment portion 131 to the treatment target.

Moreover, for example, at the time of applying the high-frequency energy to the treatment target, the control device 3 supplies the high-frequency power in between the electrode EP, which is disposed in the holder 14, and the ultrasonic blade 13 via the electric cable C. When the high-frequency power is supplied in between the electrode EP and the ultrasonic blade 13, a high-frequency current is supplied to the body tissue representing the treatment target that is being gripped in between the jaw 11 and the treatment portion 131. In other words, the high-frequency energy is applied to the treatment target.

### Regarding Configuration of Jaw

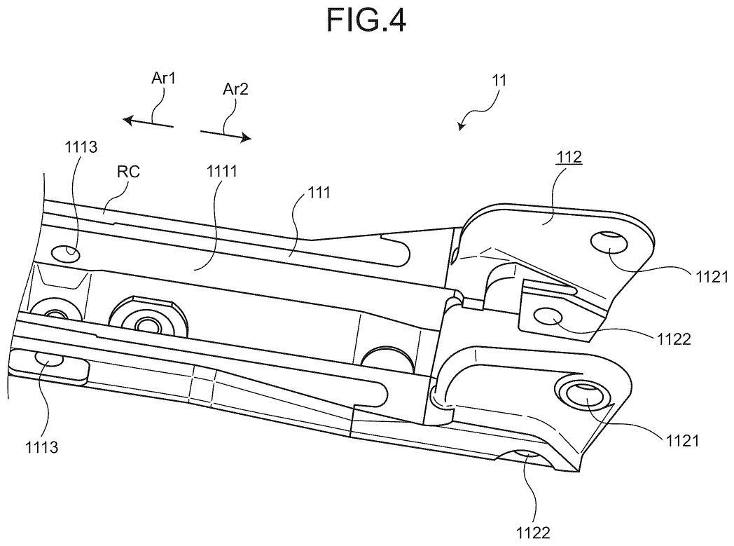

FIG. 4 is a diagram for explaining a configuration of the jaw 11. More particularly, FIG. 4 is a perspective view of the jaw 11 when viewed from the side of the treatment portion 131. In FIG. 4, for explanatory convenience, the front end portion is not illustrated.

The jaw 11 is made of an electrically conductive material. As illustrated in FIG. 4, in the jaw 11; a jaw main body 111 and a pair of bearings 112 are formed in an integrated manner.

The jaw main body 111 is made of substantially plate bodies having an elongated shape. As illustrated in FIG. 4, in the jaw main body 111, on the face present on the side of the treatment portion 131, a concave portion 1111 that extends toward the front end side Ar1 from the proximal end of the jaw main body 111 along the longitudinal direction is provided.

In the sidewall portions of the jaw main body 111 that are present on both sides in the width direction and that constitute the concave portion 1111, at the substantially central position of the jaw 11 in the longitudinal direction, a through hole 1113 is formed to pass through the front and back sides of the sidewall portions as illustrated in FIG. 4. In the through hole 1113, the third pin Pi3 is inserted and is fixed by welding.

Moreover, in the jaw main body 111, on the face of the backside present at a distance from the treatment portion 131, a cover RC (see FIGS. 2 and 4) made of an electrically insulating resin is formed in an integrated manner for covering the face of the backside of the jaw main body 111. In the first embodiment, although the cover RC is insert-molded into the jaw main body 111, that is not the only possible case. Alternatively, for example, the cover RC can be snap-fit to the jaw main body 111 or can be fixed to the jaw main body 111 using a metallic pin.

The pair of bearings 112 are disposed at the end portion of the jaw main body 111 on the proximal end side Ar2, and are made by plate bodies that are placed to face each other in the width direction of the jaw main body 111. The pair of bearings 112 have an identical configuration. Hence, the following explanation is given about the configuration of only one bearing 112.

In each bearing 112, a first insertion through hole 1121 and a second insertion through hole 1122 are formed to pass through the front and back sides of each bearing 112. When the first pin Pi1 is inserted into the first insertion through hole 1121, the bearing 112 is connected to the outer pipe 10. Moreover, when the second pin Pi2 is inserted into the second insertion through hole 1122, the bearing 112 is connected to the inner pipe PI.

### Regarding Configuration of Holder

FIG. 5 is a diagram for explaining a configuration of the holder 14. More particularly, FIG. 5 is a perspective view of the holder 14 when viewed from the side of the treatment portion 131. In FIG. 5, for explanatory convenience, dots are illustrated for the electrode EP, the first conducting surface CS1, and the second conducting surface CS2.

The holder 14 is made of a first material having electrical insulation properties and biocompatibility. For example, the first material is made of a resin such as polyetheretherketone (PEEK) or polyphenylsulfone (PPSU). Alternatively, the first material can be a material such as ceramic having electrical insulation properties. As illustrated in FIG. 5, in the holder 14; a holder main body 141, a plurality of first teeth portions 142, and a plurality of second teeth portions 143 are formed in an integrated manner.

The holder main body 141 is made of an elongated plate body. The external shape of the holder main body 141 is substantially identical to the internal shape of the concave portion 1111.

In the holder 14, the electrode EP that supplies a high-frequency current is disposed on a face that is included in the holder 14 and that faces the ultrasonic blade 13.

As illustrated in FIG. 5, the first teeth portions 142 protrude from one side in the width direction of the face of the holder main body 141 on the side of the treatment portion 131 toward the side of the treatment portion 131, and are disposed in parallel along the longitudinal direction of the holder main body 141.

As illustrated in FIG. 5, the second teeth portions 143 protrude from the other side in the width direction of the face of the holder main body 141 on the side of the treatment portion 131 toward the treatment portion 131, and are disposed in parallel along the longitudinal direction of the holder main body 141.

As illustrated in FIG. 5, on the face of the holder main body 141 on the side of the treatment portion 131, in the central portion in the width direction and in between the first teeth portions 142 and the second teeth portions 143, a concave portion 144 that is depressed toward the side away from the treatment portion 131 and that extends along the longitudinal direction of the holder main body 141 is formed. Moreover, in the holder main body 141, on the sidewall portions that constitute the concave portion 144 and that are on both sides of the holder main body 141 in the width direction, claw portions 145 and 146 (see FIGS. 3 and 5) that protrude toward the central portion in the width direction and that extend along the longitudinal direction of the holder main body 141 are disposed. When locked in the claw portions 145 and 146, the abutting portion 12 gets mechanically fixed to the holder 14. That is, the abutting portion 12 is disposed in the central portion of the holder 14 in the width direction. Furthermore, in the holder main body 141 at the sidewall portions that constitute the concave portion 144 and that are on both sides of the holder main body 141 in the width direction, an insertion through hole 147 which passes through the front and back sides of the sidewall portions and through which the third pin Pi3 is inserted is formed.

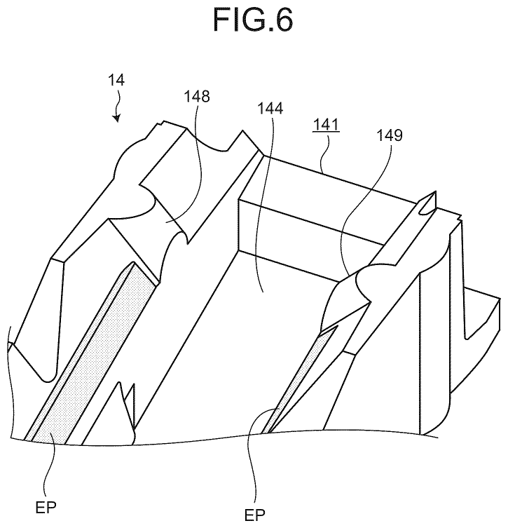

FIG. 6 is a partially expanded view of the proximal end side Ar2 of the holder 14. In the holder 14, protruding portions 148 and 149 that protrude toward the ultrasonic blade 13 are disposed. The protruding portions 148 and 149 are positioned on the proximal end side Ar2 of the holder 14 in the longitudinal direction. Moreover, the protruding portions 148 and 149 are disposed on both sides of the abutting portion 12 in the width direction. Furthermore, the protruding portions 148 and 149 are disposed to be adjacent to the electrode EP on the proximal end side Ar2 in the longitudinal direction.

Regarding the protruding portions 148 and 149, there is no particular restriction on the protruding height. Meanwhile, the protruding portions are not limited to be disposed on the proximal end side Ar2 of the holder 14 in the longitudinal direction, and alternatively can be disposed on the front end side of the holder 14 in the longitudinal direction or in the central portion of the holder 14 in the longitudinal direction.

### Regarding Configuration of Electrode, First Conducting Surface, and Second Conducting Surface

As illustrated in FIG. 5, the electrode EP is disposed on the inner side of the first teeth portions 142 in the width direction (i.e., on the side of the second teeth portions 143), is disposed on the inner side of the second teeth portions 143 (i.e., on the side of the first teeth portions 142), and is disposed in the front end portion of the holder 14 in a manner connecting the inner side of the first teeth portions 142 and the inner side of the second teeth portions 143. As a result of providing the electrode EP also in the front end portion of the holder 14, the treatment target can be treated using the front end too. However, the electrode EP need not be joined to the front end portion of the holder 14. Moreover, the electrode EP is coated with a coating material that is non-adhesive with respect to the treatment target. The coating material includes fluorine and silicon, and is extremely thin having the thickness of about a few hundred nm to few μm.

As illustrated in FIG. 5, the first conducting surface CS1 is provided on the inner face of the insertion through hole 147.

As illustrated in FIG. 5, the second conducting surface CS2 is provided on the sidewall portions of the concave portion 144, and electrically connects the electrode EP and the first conducting surface CS1 to each other.

The electrode EP, the first conducting surface CS1, and the second conducting surface CS2 are formed by performing three-dimensional plating as explained below. More particularly, a laser is irradiated at predetermined positions on the holder 14. Then, non-electrolytic plating is performed so that the electrode EP, the first conducting surface CS1, and the second conducting surface CS2 are formed at the laser irradiation positions. Herein, the electrode EP, the first conducting surface CS1, and the second conducting surface CS2 have thickness of, for example, about few μm. The formation obtained as a result of performing three-dimensional plating is called a molded interconnect device (MID) because of the fact that an electrode circuit is formed on the outer surface of a three-dimensional resin molded product. Meanwhile, the electrode EP, the first conducting surface CS1, and the second conducting surface CS2 are not limited to be formed by performing three-dimensional plating, and can be formed according to some other method.

Then, in order to apply the high-frequency energy to the treatment target, at the time of supplying the high-frequency power to the electrode EP, the high-frequency power is supplied by following the electrical pathway including the electric cable C, the outer pipe 10, the first pin Pi1, the jaw 11, the third pin Pi3, the first conducting surface CS1, the second conducting surface CS2, and the electrode EP in that order.

Regarding the Case in which the Front End Portion of the Ultrasonic Treatment Tool is Twisted

When the operator such as a technician performs treatment using the treatment tool 2, with the aim of securing the field of view, there are times when the operation handle 7 of the treatment tool 2 is rotated in the state in which the treatment target is gripped by closing the jaw 11, and the front end portion of the treatment tool 2 is twisted.

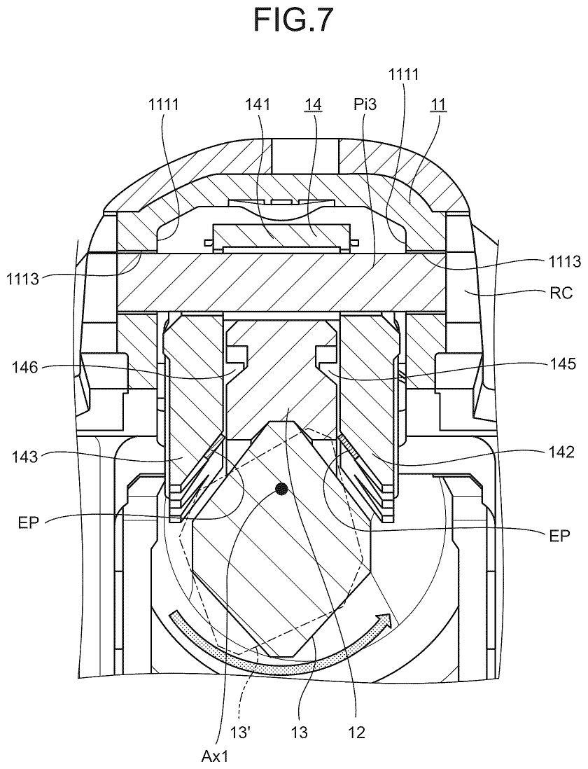

FIG. 7 is a diagram illustrating the positional relationship among the constituent elements in the case in which the front end portion of the treatment tool 2 is twisted. When the entire front end portion of the treatment tool 2 is rotated in the direction of an arrow illustrated in FIG. 7, the amount of rotation of the ultrasonic blade 13 becomes relatively smaller as compared to the amount of rotation of the holder 14. As a result, an ultrasonic blade 13′ illustrated by a dashed line in FIG. 7 becomes the relative position of the ultrasonic blade 13 with respect to the holder 14. In that positional relationship, it can be seen that the ultrasonic blade 13′ and the electrode EP move closer to each other.

On the proximal end side Ar2 of the holder 14, the protruding portions 148 and 149 are disposed. When the front end portion of the treatment tool 2 is twisted, once the protruding portions 148 and 149 abut against the ultrasonic blade 13, since the ultrasonic blade 13 rotates following the holder 14, the difference between the amount of rotation of the holder 14 and the amount of rotation of the ultrasonic blade 13 becomes smaller. As a result, it becomes possible to hold down the movement of the ultrasonic blade 13 and the electrode EP toward each other, and to hold down a situation in which the ultrasonic blade 13 and the electrode EP come in contact thereby resulting in sparks.

According to the first embodiment described above, the following effects can be achieved.

In the treatment tool 2 according to the first embodiment, since the protruding portions 148 and 149 are disposed in the holder 14, when the front end portion of the treatment tool 2 is twisted, it becomes possible to hold down a situation in which the ultrasonic blade 13 and the electrode EP come in contact thereby resulting in sparks.

Moreover, since the electrode EP is formed by performing three-dimensional plating, even when the holder 14 is made of an electrically insulating material, the electrode EP can be easily formed at a specific position on the holder 14.

Furthermore, the electrode EP is applied with a coating material that is non-adhesive with respect to the treatment target. Hence, it becomes possible to hold down the sticking of the treatment target to the electrode EP, and to favorably treat the treatment target.

### Second Embodiment

### Overall Configuration of Treatment System

A treatment system according to a second embodiment has an identical overall configuration to the configuration illustrated in FIG. 1. Hence, that explanation is not given again. The configuration that is identical to the treatment system is referred to by the same reference numerals, and the configuration that is different than the treatment system 1 is referred to by new reference numerals.

### Configuration of Treatment Tool

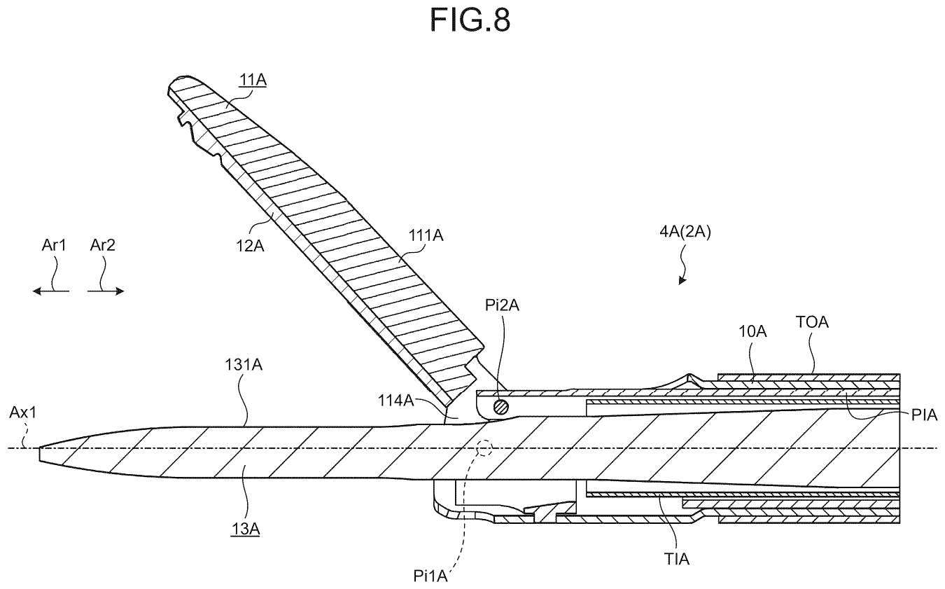

FIG. 8 is a diagram illustrating the positional relationship among the constituent elements in the case in which the front end portion of an ultrasonic treatment tool is twisted. As illustrated in FIG. 8, a handpiece 4A of a treatment tool 2A includes an outer pipe 10A, a jaw 11A, an abutting portion 12A, and an ultrasonic blade 13A.

The outer pipe 10A has a tubular shape and represents a pipe. In the second embodiment, the outer pipe 10A is a cylindrical pipe made of an electrically conductive material such as a metal.

In the outer pipe 10A, at the end portion on the front end side Ar1, a first pin Pi1A (see FIGS. 1 and 8) that has a columnar shape extending in the direction orthogonal to the plane in which FIGS. 1 and 8 are illustrated, that engages with the jaw 11, and that supports the jaw 11A in a pivotable manner is fixed. In the second embodiment, the first pin Pi1A is made of an electrically conductive material such as a metal. The first pin Pi1A represents a pin.

The outer periphery of the outer pipe 10A is covered by an outer tube TOA (see FIG. 8) that has electrical insulation properties. Inside the outer pipe 10A, a tubular inner pipe PIA (see FIG. 8) that reciprocates along the longitudinal direction of the outer pipe 10A according to an opening-closing action of the operation handle 7 performed by the operator such as a technician is inserted. Moreover, at the end portion of the inner pipe PIA on the front end side Ar1, a second pin Pi2A (see FIG. 8) that has a columnar shape extending in the direction orthogonal to the plane in which FIGS. 1 and 8 are illustrated and that engages with the jaw 11A is fixed. In the second embodiment, in FIG. 8, with reference to the first pin Pi1A, the second pin Pi2A is disposed on the upper side (i.e., on the side on which a jaw main body 111A is disposed with respect to a treatment portion 131A).

When the first pin Pi1A is inserted into the insertion through hole formed on a bearing 114A, the jaw 11A is connected to the outer pipe 10A. Moreover, when the second pin Pi2A is inserted into the insertion through hole formed on the bearing 114A, the jaw 11A is connected to the inner pipe PIA. Then, in tandem with the reciprocation of the inner pipe PIA according to an opening-closing action of the operation handle 7 performed by the operator such as a technician, the jaw 11A pivots with respect to the outer pipe 10A around the first pin Pi1A. It results in the opening and closing of the jaw 11A with respect to a treatment portion 131A representing the end portion of the ultrasonic blade 13A on the front end side, and the treatment target can be gripped in between the jaw 11A and the treatment portion 131A.

Meanwhile, in an identical manner to the first embodiment, the treatment tool 2A can be configured as a close-by-pressing type tool or as a close-by-pulling type tool. In an identical manner to the first embodiment, as the treatment tool 2A, according to an opening-closing operation of the operation handle 7, either the inner pipe PIA can reciprocate and the jaw 11A can open and close, or the outer pipe 10A can reciprocate and the jaw 11A can open and close.

The jaw 11A is made of the first material having electrical insulation properties and biocompatibility. For example, the first material is made of a resin such as polyetheretherketone (PEEK) or polyphenylsulfone (PPSU). Alternatively, the first material can be a material such as ceramic having electrical insulation properties.

The abutting portion 12A is made of the first resin material having electrical insulation properties and biocompatibility. The first resin material is, for example, polytetrafluoroethylene (PTFE). The abutting portion 12A has a substantially cuboid shape extending along the longitudinal direction of the jaw 11A. As illustrated in FIG. 8, the abutting portion 12A is fixed to the face that is included in the jaw main body 111A and that is located on the side of the treatment portion 131A; and, when the jaw 11A closes with respect to the treatment portion 131A, abuts against the treatment portion 131A. The abutting portion 12A has the function by which, when dissection of the treatment target due to ultrasonic vibrations is completed, the treatment portion 131A undergoing ultrasonic vibrations is prevented from colliding with the jaw 11A and getting damaged.

The ultrasonic blade 13A supplies ultrasonic vibrations and a high-frequency current to the body tissue. The ultrasonic blade 13A is made of an electrically conductive material and has an elongated shape extending along the central axis Ax1. As illustrated in FIG. 8, in the state in which the treatment portion 131A is protruding toward the outside, the ultrasonic blade 13A is inserted inside the inner pipe PIA. At that time, as illustrated in FIG. 1, the end portion of the ultrasonic blade 13A on the proximal end side Ar2 gets mechanically connected to the ultrasonic vibrator 52 that constitutes the ultrasonic transducer 5. The ultrasonic blade 13A transmits the ultrasonic vibrations, which are generated by the ultrasonic transducer 5, from the end portion of the ultrasonic blade 13A on the proximal end side Ar2 to the treatment portion 131A. The ultrasonic vibrations are longitudinal vibrations occurring along the central axis Ax1. Meanwhile, the outer periphery of the ultrasonic blade 13A excluding the treatment portion 131A is covered by an inner tube TIA (see FIG. 8) that is made of an electrically insulating material.

### Regarding Configuration of Jaw

In the jaw 11A, an electrode supplying a high-frequency current is disposed on a face that is included in the jaw 11A and that faces the ultrasonic blade 13A. Moreover, in the jaw 11A, protruding portions that protrude toward the ultrasonic blade 13A are disposed. The protruding portions are positioned on the proximal end side Ar2 of the jaw 11A in the longitudinal direction. Moreover, the protruding portions are disposed on both sides of the abutting portion 12A in the width direction. Furthermore, the protruding portions are disposed to be adjacent to the electrode in the longitudinal direction.

According to the second embodiment described above, the following effects can be achieved.

In the treatment tool 2A according to the second embodiment, since the protruding portions are disposed in the jaw 11A, when the front end portion of the treatment tool 2A is twisted, it becomes possible to hold down a situation in which the ultrasonic blade 13A and the electrode come in contact thereby resulting in sparks.

Moreover, since the electrode is formed by performing three-dimensional plating, even when the jaw 11A is made of an electrically insulating material, the electrode can be easily formed at a specific position on the jaw 11A.

Furthermore, the electrode is applied with a coating material that is non-adhesive with respect to the treatment target. Hence, it becomes possible to hold down the sticking of the treatment target to the electrode, and to favorably treat the treatment target.

According to the disclosure, it becomes possible to implement an ultrasonic treatment tool that enables holding down the occurrence of sparks.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic treatment tool comprising:
an ultrasonic blade configured to supply ultrasonic vibration and high-frequency current to a body tissue;
a jaw configured to open and close with respect to the ultrasonic blade;
a holder that is supported by the jaw, is made of a first material having electrical insulation properties, and includes an electrode supplying the high-frequency current and a protruding portion protruding toward the ultrasonic blade, the electrode being disposed on a face that is included in the holder and that faces the ultrasonic blade; and
an abutting portion that is disposed in the holder and is made of a first resin material, the abutting portion being configured to abut against the ultrasonic blade when the jaw closes with respect to the ultrasonic blade.

2. The ultrasonic treatment tool according to claim 1, wherein
the abutting portion is disposed in a central portion of the holder in a width direction, and
the protruding portion is disposed on each of both sides of the abutting portion in the width direction.

3. The ultrasonic treatment tool according to claim 1, wherein the protruding portion is positioned on a proximal end side of the holder in a longitudinal direction.

4. The ultrasonic treatment tool according to claim 1, wherein the first material is a resin.

5. The ultrasonic treatment tool according to claim 4, wherein the first material is polyetheretherketone or polyphenylsulfone.

6. The ultrasonic treatment tool according to claim 1, wherein the electrode is formed by performing three-dimensional plating.

7. The ultrasonic treatment tool according to claim 1, further comprising a pin that has a columnar shape and is attached to the jaw, the pin being configured to support the holder in a swingable manner, wherein
the electrode is electrically connected to the jaw via the pin.

8. The ultrasonic treatment tool according to claim 7, wherein
the holder includes an insertion through hole through which the pin is inserted, and
a first conducting surface that electrically connects the pin and the electrode to each other is disposed on an inner face of the insertion through hole.

9. The ultrasonic treatment tool according to claim 8, wherein, the holder includes a second conducting surface that electrically connects the first conducting surface and the electrode to each other.

10. The ultrasonic treatment tool according to claim 1, wherein the holder is swingable with respect to the jaw.

11. The ultrasonic treatment tool according to claim 1, further comprising:
a pin having a columnar shape; and
a pipe to which the pin is attached to the pin, the pipe being configured to support the jaw via the pin in a pivotable manner.

12. The ultrasonic treatment tool according to claim 1, wherein a coating material that is non-adhesive with respect to the body tissue is formed on the electrode.

13. An ultrasonic treatment tool comprising:
an ultrasonic blade supplying ultrasonic vibration and high-frequency current to a body tissue;
a jaw that is made of a first material having electrical insulation properties and includes an electrode supplying the high-frequency current and a protruding portion protruding toward the ultrasonic blade, the jaw being configured to open and close with respect to the ultrasonic blade, the electrode being disposed on a face that is included in the jaw and that faces the ultrasonic blade; and
an abutting portion that is disposed in the jaw, and is made of a first resin material, the abutting portion being configured to abut against the ultrasonic blade when the jaw closes with respect to the ultrasonic blade.
